(19) United States
(12) Patent Application Publication
(10) Pub. No.: US 2021/0319804 A1
(43) Pub. Date: Oct. 14, 2021

(54) SYSTEMS AND METHODS USING NEURAL NETWORKS TO IDENTIFY PRODUCERS OF HEALTH SOUNDS

(71) Applicant: University of Washington, Seattle, WA (US)

(72) Inventors: Matthew Whitehill, Seattle, WA (US); Shwetak N. Patel, Seattle, WA (US)

(73) Assignee: University of Washington, Seattle, WA (US)

(21) Appl. No.: 17/220,677

(22) Filed: Apr. 1, 2021

Related U.S. Application Data
(60) Provisional application No. 63/003,677, filed on Apr. 1, 2020.

(51) Int. Cl. G10L 25/66; G10L 25/30; G10L 17/18; G10L 21/10; G06N 3/08

(52) U.S. Cl. CPC G10L 25/66; G10L 25/30; G06N 3/08; G10L 21/10; G10L 17/18

(57) ABSTRACT

Examples of apparatuses and methods described herein may provide personalized audio health sensing to identify individuals based on their health sounds. A microphone may receive an audio sample including speech utterance and cough. A computing device may process the audio sample and analyze the audio sample to predict whether the audio sample is produced by a known user. The computing device may include a neural network that processes and analyzes the audio sample.

### CROSS-REFERENCE TO RELATED APPLICATIONS

This application claims the benefit under 35 U.S.C. 119(e) of the earlier filing date of U.S. Provisional Application No. 63/003,677 filed on Apr. 1, 2020. The aforementioned application is all incorporated herein by reference, in its entirety, for any purpose.

### TECHNICAL FIELD

Examples described herein relate generally to identifying a health sound producer. Examples of identification using a computing device and a neural network are described.

### BACKGROUND

Automatic cough counting systems have served to determine how many coughs are present in an audio recording. However, they cannot determine who produced the cough. This limits their usefulness as most systems are deployed in locations with multiple people (e.g., a smart home device in a four-person home).

Existing cough counting algorithms face one important limitation they cannot identify who produced the cough. This means whenever multiple people inhabit a common space, cough counting algorithms cannot attribute a cough to the right person. Thus, identifying who produced a cough sample could dramatically increase the utility of these systems.

In comparison with the existing speaker verification systems, examples described herein may provide significant advantages in terms of accuracy of coughs.

### BRIEF DESCRIPTION OF THE DRAWINGS

FIG. 1 is a schematic illustration of a computing device arranged in accordance with examples described herein.

FIG. 2 is a schematic illustration of the multi-task training of an encoder in accordance with examples described herein.

FIG. 3 is a schematic illustration of audio data processing with a model architecture in accordance with examples described herein.

FIG. 4 is a schematic illustration of an example architecture for the encoder in accordance with examples described herein.

FIG. 5 is a block diagram of example computing network arranged in accordance with an example embodiment,

FIG. 6 is a block diagram of example computing device arranged in accordance with examples described herein.

FIG. 7 depicts visualizations of t-SNE clustering of cough embeddings for one cross-validation test in accordance with examples described herein.

### DETAILED DESCRIPTION

Examples of technology described herein include systems and methods that can provide personalized audio health sensing. For example, certain health sounds, such as sounds made by biological processes, may be analyzed and associated with a particular individual (e.g., the individual producing the sound). In this manner, systems and methods described herein may be able to identify individuals based on their health sounds.

Examples of methods described herein may include obtaining health sound audio, encoding the health sound audio to obtain an alternate representation, comparing the alternate representation to an alternate representation of enrolled health sound audio samples from known users, and associating a selected one of the known users to the health sound audio based on the comparison.

Examples of systems and methods described herein may be used to associate individuals with any of a variety of health sounds. Examples of health sounds include, but are not limited to, coughs, burps, flatulence, snores, sneezes, sniffles, wheezes, throat clearing, or expectorating. These health sounds may differ from speech because of their particular structure (e.g. coughs have an explosive phase, exhale, and occasionally voiced phase). This structure may facilitate successful methods in identifying the user who produced a health sound that is different from the methods used to identify the user who produced a certain speech sample.

While examples of technology described herein could be utilized to process a wide array of health sounds, examples of systems and methods that may detect and identify individuals based on coughs are specifically described. It is to be understood that other health sounds may be used in other examples.

Coughing is a symptom of many respiratory ailments such as asthma, tuberculosis, and cystic fibrosis. Thus, counting and analyzing coughs can serve as an important diagnostic tool for these conditions. Automated cough detection systems count the number of coughs in an audio file by distinguishing them from other sounds such as speech, background noise, and music. They usually begin by converting the audio waveform to a frequency representation, then use machine learning to identify the cougher. By including the ability to identify the individual making the health sounds, examples described herein may collect health sounds from shared spaces (e.g., homes, classrooms, hospitals, buildings) where multiple individuals may be present and making health sounds. The health sounds belonging to a particular individual may be identified (e.g., labeled) such that they may be reviewed, transmitted, stored, and/or analyzed.

Certain details are set forth herein to provide an understanding of described embodiments of technology. However, other examples may be practiced without various of these particular details. In some instances, well-known circuits, control signals, timing protocols, and/or software operations have not been shown in detail in order to avoid unnecessarily obscuring the described embodiments. Other embodiments may be utilized, and other changes may be made, without departing from the spirit or scope of the subject matter presented here.

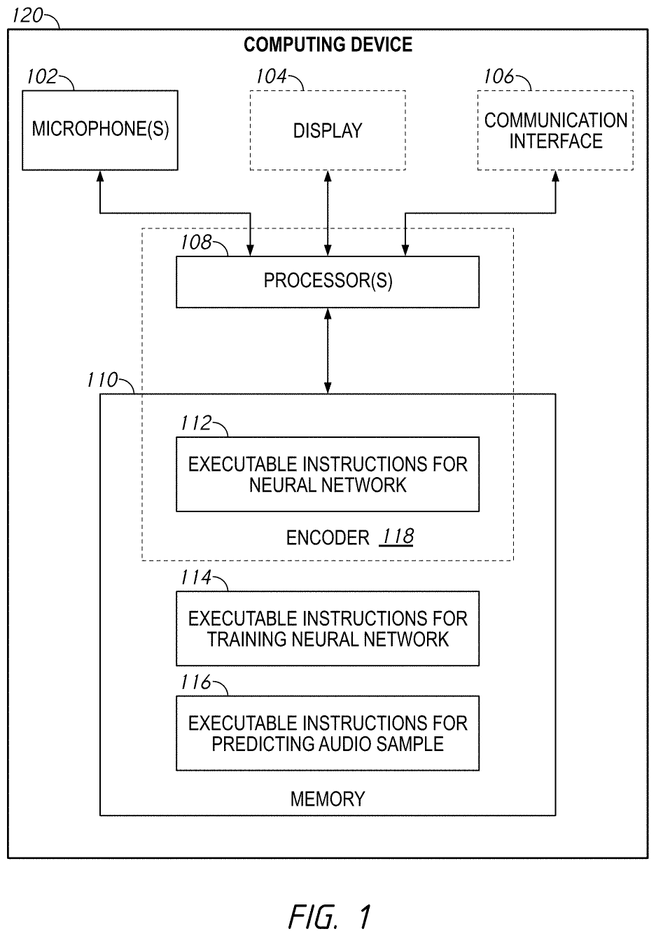

FIG. 1 is a schematic illustration of a computing device arranged in accordance with examples described herein. The computing device 120 includes microphone(s) 102 that obtains audio signals. The computing device 120 includes display 104, processor(s) 108, and a memory 110. The memory 110 includes executable instructions for neural network 112, executable instructions for training neural network 114, and executable instructions for predicting audio sample 116. Additional, fewer, and/or different components may be present in other examples. For example, the computing device 120 may include one or more communication interface(s) 106, one or more display(s) 104, additional memory and/or electronic storage, and/or additional storage. The computing device 120 may include an encoder 118 that includes the processor(s) 108 and the executable instructions for neural network 112 for executing instructions to encode or be the neural network. The processor(s) 108 may execute instructions stored in memory 110 and/or in other computer readable media accessible to the computing device 120 and/or processor(s) 108 to identify the cougher and train the neural network.

Examples of systems described herein may accordingly include computing devices. Computing device 120 is shown in FIG. 1. Generally, a computing device may include a smartphone and any electronic device in communication with a with a microphone as described herein and with one or more processors and/or communication interfaces to conduct the identification described herein to identify the cougher as described herein. Additionally or alternatively, the electronic device may also be used for and/or in communication with one or more processors to train a neural network with the audio signals. A computing device may or may not have cellular phone capability, which capability may be active or inactive. While cellular devices are described, examples of techniques described herein may be implemented in some examples using other electronic devices such as, but not limited to, tablets, laptops, smart speakers, computers, wearable devices (e.g., smartwatch), appliances, or vehicles. Generally, any device having a microphone and processor(s) may be used.

Computing devices described herein may include one or more processors, such as processor(s) 108 of FIG. 1. Any number or kind of processing circuitry may be used to implement processor(s) 108 such as, but not limited to, one or more central computing units (CPUs), graphical processing units (GPUs), logic circuitry, field programmable gate arrays (FPGAs), application specific integrated circuits (ASICs), controllers, or microcontrollers. While certain activities described herein may be described as performed by the processor(s) 108 it is to be understood that in some examples, the activities may wholly or partially be performed by one or more other processor(s) which may be in communication with processor(s) 108. That is, the distribution of computing resources may be quite flexible and the computing device 120 may be in communication with one or more other computing devices, continuously or intermittently, which may perform some or all of the processing operations described herein in some examples.

Computing devices described herein may include memory, such as memory 110 of FIG. 1. While memory 110 is depicted as, and may be, integral with computing device 120, in some examples, the memory 110 may be external to computing device 120 and may be in communication with processor(s) 108 and/or other processors in communication with processor(s) 108. While a single memory 110 is shown in FIG. 1, generally any number of memories may be present and/or used in examples described herein, Examples of memory which may be used include read only memory (ROM), random access memory (RAM), solid state drives, and/or SD cards.

Computing devices described herein may operate in accordance with software (e.g., executable instructions stored on one or more computer readable media, such as memory, and executed by one or more processors). Examples of software may include executable instructions for predicting audio sample 116, executable instructions for training neural network 114, and/or executable instructions for neural network 112 of 1. For example, the executable instructions for predicting audio sample 116 may provide instructions and/or settings for converting an audio sample recorded by microphone(s) 102 into a feature set that includes features such as the frequency representation (e.g., short-term Fourier spectrogram, Mel-frequency spectrogram, gammatone spectrogram, or cepstral coefficient spectrogram). The frequency representation of the audio sample may be encoded and used to compare with known audio data of enrolled users. The executable instructions for predicting audio sample 116 may segment the frequency representation to create an embedding, which is used to predict whether the audio sample is from an enrolled user. The display 104 may be coupled to the processor and display the result of the prediction.

Examples described herein may provide one or more neural network models. Generally, a neural network model may use components such as fully-connected layers, convolutional layers, recurrent layers, time-delay layers, residual connections, and attention layers, and statistical-pooling layers (e.g., zeroth, first, or second-order statistics). The alternate representation may be in a variety of forms such as a 1-dimensional vector, matrix, or multi-dimensional matrix.

Executable instructions for neural network 112 may include instructions and/or settings for using a neural network to encode the cough audio sample for prediction of whether the audio sample is from an enrolled user. The comparison of the alternate representation health sound audio sample to the alternate representation of enrolled health sound audio samples may be accomplished using a comparison method including but not limited to a distance metric (e.g., Euclidean distance or cosine similarity), a machine learning algorithm (e.g. linear regression, support-vector machine, principal component analysis, linear discriminant analysis, probabilistic liner discriminant analysis), or deep learning architectures including fully-connected layers, convolutional layers, recurrent layers, time-delay layers, residual connections, and attention layers, lii some examples, the computing device 120 further includes an encoder 118 that encodes the cough audio sample with processor(s) 108 and/or a neural network that follows the executable instructions for neural network 112. In some examples, the executable instructions for neural network 112 may be used together with the executable instructions for predicting audio sample 116, For example, the executable instructions for neural network 112 may be used to implement a neural network which may generate one or more embeddings based on audio input—the embeddings may be used to identify (e.g., classify or predict) the individual making any particular health noise in the audio input. Embeddings may be a single dimensional array that are a compressed representation of the sound. In some examples, embeddings may encode the most important or descriptive features of the sound. For example, neural networks described herein may be trained to create the embeddings such that the health sounds may be efficiently and accurately associated with an enrolled user. The embeddings may represent aspects of the sound that are most important for differentiating between different users. For example, sound embeddings may encode elements such as the characteristics of a cough (e.g., whether the cough is wet or dry or contains a wheeze, etc.), the fundamental frequency and other resonant frequencies or formants, and the length of phases of the cough (e.g., explosive, exhale, or—voiced phases, etc.).

Accordingly, one or more neural networks may be used herein to identify (e.g., classify or predict) the individual producing a health sound. Generally, a neural network generally refers to a collection of nodes which may be provided in layers. Each node may be connected at an input to a number of nodes from a previous layer and at an output to a number of nodes of a next layer. Generally, the output of each node may be a non-linear function of a combination (e.g., a sum) of its inputs. Generally, the coefficients used to conduct the non-linear function (e.g., to implement a weighted combination) may be referred to as weights. The weights may in some examples be an output of a neural network training process. Examples of neural networks which may be implemented and used herein include fully convolutional neural networks (e.g., neural networks having no fully connected and/or dense layers). In some examples, one or more residual neural networks (ResNets) may be used. In some examples, certain nodes of a residual neural network may utilize skip connections to jump over layers (e.g., an input and/or an output of a node may be connected to a layer not immediately adjacent the node). In some examples, one or more recurrent neural networks (RNNs) may be used. Generally, recurrent neural networks may process signals based in part on an internal state (e.g., memory) of the network. Recurrent neural networks may include feedback from one or more nodes to a previous layer.

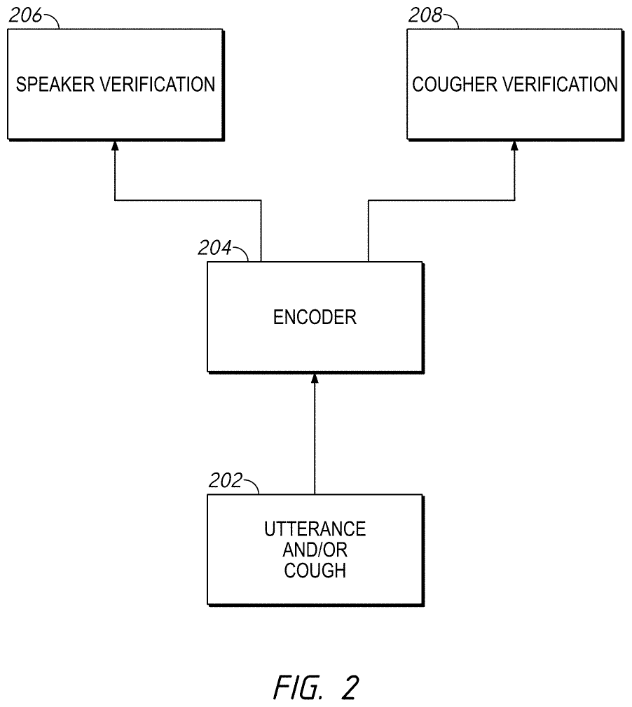

The executable instructions for training neural network 114 may include instructions and/or settings for training the neural network. A variety of training techniques may be used—including supervised and/or unsupervised learning. In some examples, a same computing device may be used to train the neural network (e.g., may implement executable instructions for training neural network 114) as used to classify the individual making the health sound. In other examples, a different computing device may be used to train the neural network and output of the training process (e.g., weights, connections, and/or other neural network specifics) may be communicated to and/or stored in a location accessible to the computing device used to classify the individual making the health sound. In some examples, the executable instructions for training neural network 114 may include instructions for multi-task learning. Multi-task learning generally refers to training the model utilizing multiple tasks. For example, the executable instructions for training neural network 114 may including instructions for training on both audio data include health sounds (e.g., coughs) and speech. By training on both health sounds and on speech, in some examples, the model's performance may be improved relative to a model trained only on health sounds and/or only on speech, Additional details of multitask learning in the model are discussed herein, including with respect to FIG. 2.

Generally, training may utilize training data. For example, training data may include a variety of audio data including coughs made by various users. The neural network may be trained to generate embeddings for the health sounds such that coughs made by same users are classified to a same user, while a cough made by a different user is accurately distinguished by the neural network. Multi-task learning may be used for example the neural network may also be trained using training speech data and adjusted such that speech samples from the same user are classified to the same user, while speech made by a different user is accurately distinguished.

During operation, the microphone(s) 102 may receive audio data including one or more health sounds. The encoder 118 may encode the health sounds into one or more embeddings, Note that, while FIG. 1 depicts the encoder 118 in a same computing device as receives the audio data including health sounds (e.g., in the same computing device as microphone(s) 102), in some examples, the encoder 118 may be implemented in a different computing device and may receive transmitted and/or stored audio data received at a microphone of another device.

The encoder 118 may encode the health sounds into one or more embeddings. To encode the health sounds, the encoder 118 may utilize a neural network (e.g., executable instructions for neural network 112). The neural network in some examples may be a trained neural network (e.g., a neural network trained using multi-task learning techniques), The trained neural network may be trained to generate embeddings based on audio data which are particularly efficient and/or accurate at classifying health sounds. In some examples, framing may be used, such that the encoder 118 may generate an embedding for each of multiple frames and/or subsets of the audio data for a health sound. The use of framing may assist in accurate representation of a health sound having a particular structure (e.g., a cough). One or more embeddings may be generated for each phase of the structured health sound (e.g., in an example using a cough, one or more embeddings may be generated for the inspiratory phase, the compressive phase, and the expiratory phase). In some examples, the multiple embeddings may be combined to provide a global embedding. The global embedding may be compared (e.g., using executable instructions for predicting audio sample 116) to enrolled users. For example, an enrolled user may have one or more stored embeddings corresponding to known speech and/or health sounds made by that user and processed by a neural network (e.g., executable instructions for neural network 112 or similar), A comparison of the new global embedding may be made to enrolled users, and the individual producing the health sound may be identified (e.g., predicted or classified).

In this manner, examples of computing devices described herein may identify an individual producing a health sound (e.g., also referred to herein as the speaker and/or the tougher) based on audio data. Examples of an implemented neural network model described herein achieved 82.15% classification accuracy amongst four users on a natural, in-the-wild cough dataset, outperforming human evaluators on average by 9.82%.

Examples of computing devices, systems, and techniques described herein may find a variety of uses. For example, one or more users may review their own history of health sounds. For example, health sounds predicted and/or identified to be from a particular user may be stored together with an association to that user (e.g., in memory 110 or in another memory or storage). For example, one or more users may track their own cough in a variety of clinical conditions such as diagnosing the root cause of chronic cough (e.g., associating the time of day the user(s) coughs may be tied to the type of health condition), predicting Chronic Obstructive Pulmonary Disease (COPD) exacerbations, and monitoring the health of the user(s) with an acute respiratory infection such as COVID-19. For example, when more than one person coughs in a room, cough counts may be identified between the multiple coughers. Example of computing devices, systems, and techniques described herein may allow accurate cough counting for all of the above clinical applications. Additional information about the health sound may also be stored (e.g., the time, the volume, the frequency, the location). A user (e.g., the producer of the health sounds, a medical professional, a family member), may then review the history of health sounds made by an individual and collected by a single computing device or aggregated data from multiple collection points (e.g., computing devices at home and work). In this manner, the user may review if the frequency of health sounds increases or decreases over time or in a particular place, and may identify triggers for the health sound for that individual in some examples. In some examples a health professional may monitor health sounds (e.g., chronic cough) and may diagnose a disease related to the health sounds. A treatment may be prescribed and/or modified based on increasing or decreasing frequency of coughing, for example. In some examples, the audio data associated with the health sound may also be stored, and a user may replay the health sound.

FIG. 2 is a schematic illustration of a multitask learning training of an encoder in accordance with examples described herein. FIG. 2 depicts encoder 204 which may receive audio sample including utterances and/or coughs 202. In some examples, the encoder 204 may be implemented by the encoder 118, processor(s) 108, or executable instructions for neural network 112 of FIG. 1, In some examples, the executable instructions for training neural network 114 may include executable instructions for performing the actions described with respect to speaker verification 206 and cougher verification 208 of FIG. 2. For example, techniques shown and described with reference to FIG. 2 may in some examples be used to train the neural network implemented by executable instructions for neural network 112 of FIG. 1. An output of the training may be, for example, weights, which may be in some examples stored in memory 110 of FIG. 1 and/or otherwise accessible to the processor of FIG. 1 and used when executing the executable instructions for neural network 112.

The components in FIG. 2 are examples only, Additional, fewer, and/or different components may be used in other examples. While the example of FIG. 2 is shown and described in the context of coughs, it is to be understood that other health sounds may additionally or instead be verified in cougher verification 208.

The encoder 204 may encode an input audio sample and process the utterances and/or coughs 202 in the audio sample by performing speaker verification 206 based on the test utterances and performing cougher verification 208 based on the coughs.

Speaker verification 206 involves determining whether a speech segment (utterance) comes from an enrolled user. A speaker is initially enrolled when his or her utterance is processed through the encoder 204 to produce an embedding. Next, speakers are enrolled by aggregating known utterances from the specific speaker. Test utterances from the audio sample may then be compared to the known utterances. If the test utterance is similar enough to the enrolled speaker, the speaker is verified to be an enrolled speaker.

In some examples, the encoder 204 may encode the audio sample to obtain an alternate representation which is compared to enrolled health sound audio samples for predicting whether the audio sample is from an enrolled user.

Cougher verification 208 involves comparing coughs of a user to existing cough data of enrolled users. In some examples of enrollment, cougher verification 208 may be trained with forced coughs, where a user instructed to produce a cough. However, forced coughs between different users may sound similar because the participants consciously think about coughing. Unlike speech, certain audio health sounds have a predictable particular structure. For example, a natural cough is typically comprised of an explosive phase, an exhale, and occasionally a voiced component. Other sounds that also have particular structures include burps, flatulence, snores, sneezes, sniffles, wheezes, throat clearing, or expectorating. In some examples, methods may be used to take advantage of this structure such as pattern-matching to a library of structures, identifying each phase in the audio sample or features and utilizing the features associated with each phase differently, or using a sequential encoding component such as a recurrent neural network layer (e.g., GRU, LSTM).

Examples described herein may train a speaker verification model implemented by speaker verification 206 and test the model on a natural cough dataset. In some examples, the speaker verification model may be a sound detector and bring together audio samples that sound similar to humans but are from a different domain (coughs). The speaker verification model may learn from the sound detector and create generalizable features from the cougher verification task.

In some examples, the encoder 204 may utilize deep neural networks to produce speaker embeddings. In some examples, a 3-layer long short-term memory (LSTM) network (e.g., a recurrent neural network) and an end-to-end trainable cosine similarity metric may be used to compare utterance embeddings, using cosine similarity, but with siamese training and a resnet-inspired convolutional neural network to create the embeddings, and a time-delay neural network and statistical pooling layer to create embeddings called x-vectors, then a probabilistic linear discriminant analysis (PLDA) classifier to compare x-vectors.

For example, the encoder 204 may utilize generalized end-to-end loss. It is to be understood that alternative losses (e.g., Siamese training) may be used. Let eᵢⱼ be the output embedding where i is the speaker or cougher ID and j is the utterance or cough ID. Let cₖ be the centroid of all embeddings for the speaker or cougher k; however, where i=eⱼₖ is removed from the centroid calculation of dc. The similarity matrix Sᵢⱼ is the cosine similarity from the embedding eᵢⱼ to each centroid cₖ:

S_{i,j,k}=w·cos*eᵢⱼ,cₖ)+b

where cos is the cosine similarity function and w and b are learnable parameters. Softmax may be used to calculate the loss:

$${L}_{n} = {\sum }_{ij} ( - {S}_{ij , i} + log {\sum }_{k = 1}^{N} exp ( {S}_{ij , k} )$$

where N is the number of speakers or coughers and n is the task number. The full loss is

L=L₁+∝L₂

where L₁ is the speaker verification task and L₂ is the cougher verification task. In some examples where there may be fewer coughers than speakers, 0.05 may be used for α to encourage progress on the speaker task before focusing on the cougher task. The learnable parameters (e.g., w and b) may be used as outputs of the training process. In some examples, the above 3-layer LSTM network may be used, with the learnable parameters determined during training, to implement the encoder of FIG. 1.

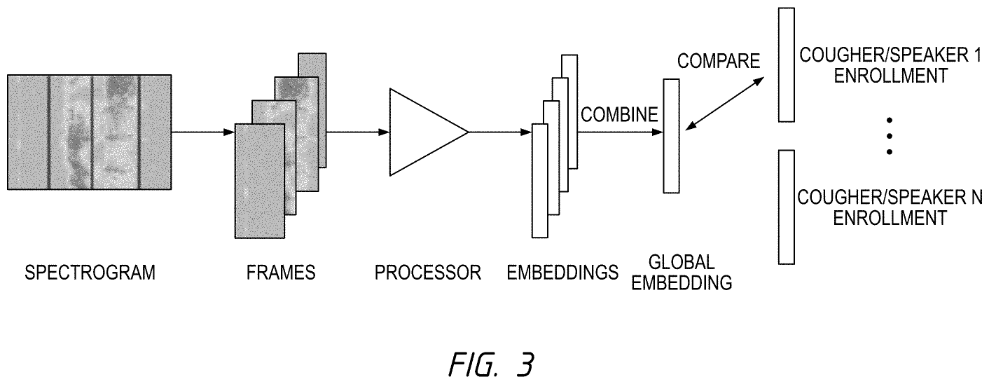

FIG. 3 is a schematic illustration of audio data processing with a neural network in accordance with examples described herein. The audio data processing shown and described with respect to FIG. 3 may be implemented, for example, using the processor(s) 108 of FIG. 1 executing the executable instructions for neural network 112, executable instructions for training the neural network 114, and/or executable instructions for predicting audio sample 116. While examples of the audio processing shown in FIG. 3 may be used with a multi-task learning training scheme shown in FIG. 2, it is to be understood that the multitask learning training scheme is not required for use with the audio data processing of FIG. 3.

Audio data received is shown as a spectrogram in FIG. 3. Any of a variety of spectrograms may be used, and the executable instructions for predicting audio sample 116 of FIG. 1 may include instructions for generating a spectrogram from input audio. In some examples, the audio signals may be converted to a mel-frequency spectrogram. In other examples alternative feature sets could be used such as the short-term fourier spectrogram or ceptral coefficient spectrogram.

In some examples, a segment of audio data (e.g., input audio signals) may be divided into frames. In other examples, framing may not be used, Next, the spectrogram may be framed although algorithms without framing could also be used. For example, cough episodes may be of variable length, ranging from as short as 150 ms to up to 3 s, Because the same user can produce coughs that are both long and short, a frame-based approach may be utilized to prevent the model from using sample length as a feature. The audio sample may be converted to its mel-frequency representation. The spectrogram may be segmented into shorter frames. Frames of any length may be used, but in some examples, one or more frames may be used which cover a particular phase of a health sound (e.g., phase of a cough). In some examples, a Hamming window of size 25 ms with 10 ms step size and 40 mel-filterbanks may be used. Each sample is framed by stacking non-overlapping windows of 19 frames in time by the 40 mel-filterbanks. In an example, 19 frames may be selected since it is the smallest symmetric window that provided sufficient temporal dimensionality at the final encoder convolutional output.

The framed spectrogram may then be processed by an encoder to create a global embedding. The encoder may be implemented by encoder 118 of FIG. 1 and encoder 204 of FIG. 2. In some examples, the global embedding may be compared with embeddings associated with enrolled speakers by cosine distance to predict whether the sample is from an enrolled user. In other examples, alternative comparison methods could be considered such as different distance metrics, machine learning algorithms, or neural network architectures.

Each frame may be processed through a processor (e.g., a neural network) to create an embedding. In some examples, the processor may be implemented by the processor(s) 108 or encoder 118 of FIG. 1. In other examples, the processor may be implemented by the encoder 204 of FIG. 2. The processor may implement a neural network trained to generate an embedding which may be efficiently and/or accurately identified with a speaker. Generally, an embedding refers to a representation of the frame which may include particular features of the frame, combinations of features, or other information about the frame. The embeddings from each frame may be combined (e.g., averaged, weighted summed) to obtain a global embedding. In some examples, the embeddings may be averaged to obtain the global embedding. In some examples, variable length samples may be processed using recurrent neural network layers (e.g., GRU, LSTM) or pooling layers (e.g. average pooling).

In examples described herein, a residual-network (ResNet) architecture for the encoder may be used. It is to be understood that other neural network architectures (e.g., convolutional neural network) could be used for the encoder.

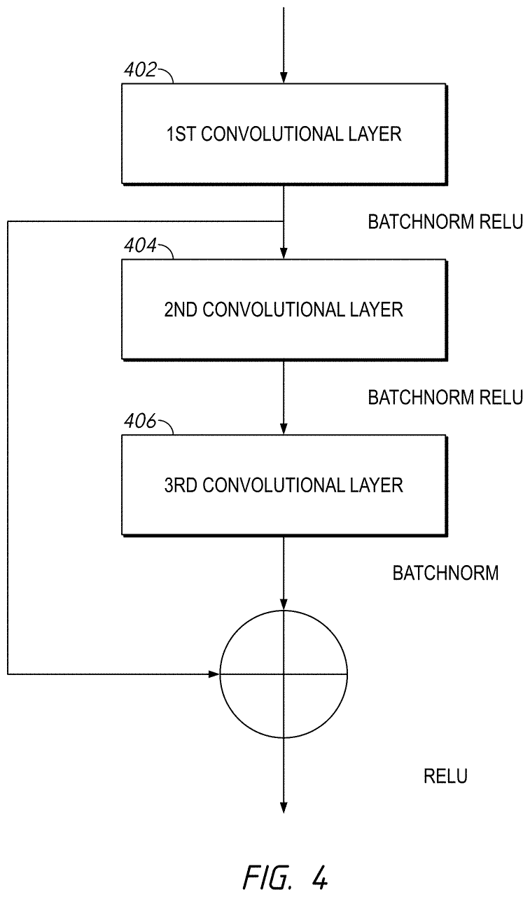

FIG. 4 illustrates an example of a residual neural network (ResNet). The residual neural network of FIG. 4 may be used to implement, for example, the encoder 118 of FIG. 1, the encoder 204 of FIG. 2, and/or the processor of FIG. 3. For example, the executable instructions for neural network 112 may include instructions for implementing the residual neural network of FIG. 4. The residual neural network of FIG. 4 includes 3 residual network blocks where each block has the same structure. The 1st convolutional layer 402 may be a 2×2 kernel and a stride of 2. The 2nd convolutional layer 404 and 3rd convolutional layer 406 may each be a 3×3 kernel and a stride of 1. In some examples, the 1st convolutional layer 402 may use 64 filters. The 2nd convolutional layer 404 may use 128 filters. The 3rd convolutional layer 406 may use 256 filters. Other numbers of filters may also be used in each of these convolutional layers. Batch normalization (batch-norm) and a rectified linear unit (Ram) may follow each convolutional layer. A skip connection links the output of the first convolutional layer's batch-norm to the output of the final layer's batch-norm. After the residual blocks, a channel-wise average pooling layer may be applied, followed by a fully-connected layer to create an embedding for each frame of input data. These embeddings may be combined or averaged to create a global embedding as described in FIG. 3.

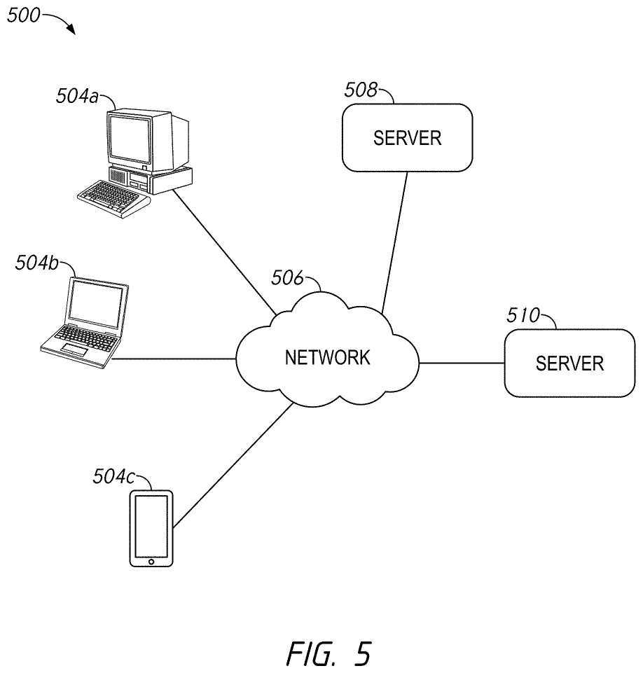

FIG. 5 is a block diagram of example computing network 500 in accordance with an example embodiment. In FIG. 5, servers 508 and 510 are configured to communicate, via a network 506, with client devices 504a, 504h, and 504c. As shown in FIG. 5, client devices can include a personal computer 504a, a laptop computer 504b, and a smart-phone 504c. More generally, client devices 504a-504c (or any additional client devices) can be any sort of computing device, such as a workstation, network terminal, desktop computer, laptop computer, wireless communication device (e.g., a cell phone or smart phone), and so on. In particular, some or all of client devices 504a-504c can collect and process of data associated with a cough detection device as disclosed herein, as well as the device in which such cough detection is implemented or implemented in part. For example, some or all of client devices 504a-504c may include a microphone to detect cough. In many embodiments, clients 504 can perform most or all of the herein-described methods. In some examples, client devices 504a-504c may be implemented by and/or used to implement computing device 120 of FIG. 1.

The network 506 can correspond to a local area network, a wide area network, a corporate intranet, the public Internet, combinations thereof, or any other type of network(s) configured to provide communication between networked computing devices. In some embodiments, part or all of the communication between networked computing devices can be secured.

Servers 508 and 510 can share content and/or provide content to client devices 504a-504c. As shown in FIG. 5, servers 508 and 510 are not physically at the same location. Alternatively, recipe servers 508 and 510 can be co-located, and/or can be accessible via a network separate from network 506. Although FIG. 5 shows three client devices and two servers, network 506 can service more or fewer than three client devices and/or more or fewer than two servers. In some embodiments, servers 508, 510 can perform some or all of the herein-described methods. For example, servers 508 and/or 510 may be used to implement and/or may be implemented using computing device 120 of FIG. 1.

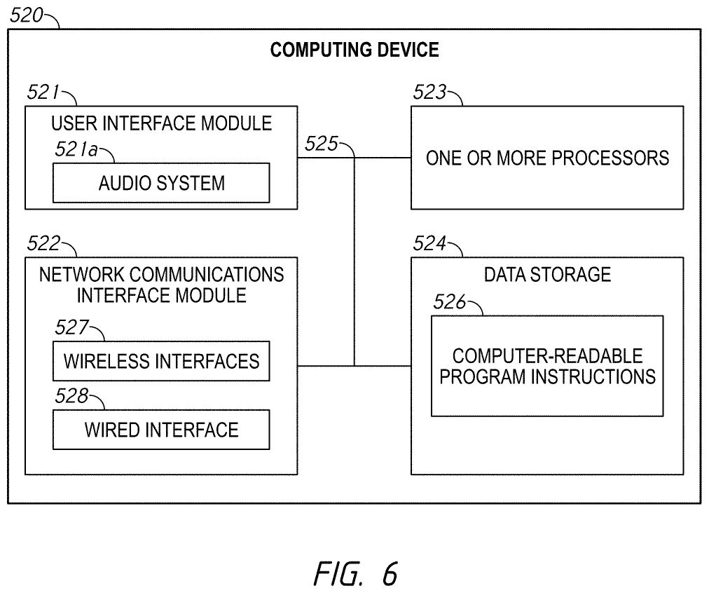

FIG. 6 is a block diagram of an example computing device 520 including user interface module 521, network-communication interface module 522, one or more processors 523, and data storage 524, in accordance with embodiments of the invention.

For example, computing device 520 shown in FIG. 6 can be configured to perform one or more functions of a system, client devices 504a-504c, network 506, and/or servers 508, 510. Computing device 520 be used to implement the computing device 120 of FIG. 1. Computing device 520 may include a user interface module 521, a network-communication interface module 522, one or more processors 523, and data storage 524, all of which may be linked together via a system bus, network, or other connection mechanism 525.

Computing device 520 can be a desktop computer, laptop or notebook computer, personal data assistant (PDA), mobile phone, video game console, embedded processor, touchless-enabled device, or any similar device that is equipped with at least one processing unit capable of executing machine-language instructions that implement at least part of the herein-described cougher verification techniques and methods. In many embodiments, computing device 520 may be implemented using a smartphone.

User interface module 521 can receive input and/or provide output, perhaps to a user. User interface module 521 can be configured to send and/or receive data to and/or from user input from input device(s), such as a microphone, a keyboard, a keypad, a touch screen, a computer mouse, a track ball, a joystick, camera, and/or other similar devices configured to receive input from a user of the computing device 520. In some embodiments, input devices can include gesture-related devices, such a video input device, a motion input device, time-of-flight sensor, RGB camera, or other 3D input device. User interface module 521 can be configured to provide output to output display devices, such as one or more cathode ray tubes (CRTs), liquid crystal displays (LCDs), light emitting diodes (LEDs), displays using digital light processing (DLP) technology, printers, light bulbs, and/or other similar devices capable of displaying graphical, textual, and/or numerical information to a user of computing device 520. User interface module 521 can also be configured to generate audible output(s), such as a speaker, speaker jack, audio output port, audio output device, earphones, and/or other similar devices configured to convey sound and/or audible information to a user of computing device 520. As shown in FIG. 6, user interface can be configured with audio system 521a that includes a microphone (e.g., audio system 521a may be used to implement and/or may be implemented by microphone(s) 102 of FIG. 1). The microphone may capture cough data discussed elsewhere herein. In embodiments, the functions of the audio system may be performed on a separate and/or remote device in communication with client device 520.

Network-communication interface module 522 can be configured to send and receive data over wireless interface 527 and/or wired interface 528 via a network, such as network 506. Wireless interface 527 if present, can utilize an air interface, such as a Bluetooth®, Wi-Fi®, ZigBee®, and/or WIMAX™ interface to a data network, such as a wide area network (WAN), a local area network (LAN), one or more public data networks (e.g., the Internet), one or more private data networks, or any combination of public and private data networks. Wired interface(s) 528, if present, can comprise a wire, cable, fiber-optic link and/or similar physical connection(s) to a data network, such as a WAN, LAN, one or more public data networks, one or more private data networks, or any combination of such networks.

In some embodiments, network-communication interface module 522 can be configured to provide reliable, secured, and/or authenticated communications. Communications can be made secure (e.g., be encoded or encrypted) and/or decrypted/decoded using one or more cryptographic protocols and/or algorithms, such as, but not limited to, DES, AES, RSA, Diffie-Hellman, and/or DSA. Other cryptographic protocols and/or algorithms can be used as well as or in addition to those listed herein to secure (and then decrypt/decode) communications.

Processor(s) 523 can include one or more central processing units, computer processors, mobile processors, digital signal processors (DSPs), microprocessors, computer chips, and/or other processing units configured to execute machine-language instructions and process data. Processor(s) 523 can be configured to execute computer-readable program instructions 526 that are contained in data storage 524 and/or other instructions as described herein.

Data storage 524 can include one or more physical and/or non-transitory storage devices, such as read-only memory (ROM), random access memory (RAM), removable-disk-drive memory, hard-disk memory, magnetic-tape memory, flash memory, and/or other storage devices. Data storage 524 can include one or more physical and/or non-transitory storage devices with at least enough combined storage capacity to contain computer-readable program instructions 526 and any associated/related data structures.

Computer-readable program instructions 526 and any data structures contained in data storage 524 include computer-readable program instructions executable by processor(s) 523 and any storage required, respectively, to perform at least part of herein-described methods for cough verification (e.g., executable instructions for neural network 112, executable instructions for training neural network 114, and executable instructions for predicting audio sample 116 of FIG. 1).

From the description herein it will be appreciated that, although specific embodiments have been described herein for purposes of illustration, various modifications may be made while remaining with the scope of the claimed technology.

Examples described herein may refer to various components as “coupled” or signals as being “provided to” or “received from” certain components. It is to be understood that in some examples the components are directly coupled one to another, while in other examples the components are coupled with intervening components disposed between them. Similarly, signal may be provided directly to and/or received directly from the recited components without intervening components, but also may be provided to and/or received from the certain components through intervening components.

### IMPLEMENTED EXAMPLES

Experimental Setup

Cough Dataset

In examples described herein, the in-the-wild, natural cough dataset on cough counting may be used. To collect the data, participants with a frequent cough carried a smartphone in their shirt pocket or on a lanyard around their neck for 3 to 6.5 hours. The dataset contains 2,445 individual coughs within 1,331 cough episodes from 16 participants (8 male, 8 female). See Table 1 for coughs per user.

TABLE 1

| User | Coughs |
|---|---|
| 1 | 32 |
| 2 | 41 |
| 3 | 53 |
| 4 | 64 |
| 5 | 67 |
| 6 | 77 |
| 7 | 79 |
| 8 | 81 |
| 9 | 85 |
| 10 | 98 |
| 11 | 102 |
| 12 | 129 |
| 13 | 142 |
| 14 | 230 |
| 15 | 261 |
| 16 | 904 |

Each individual cough is manually segmented to the beginning and end of the cough. During training, each individual cough is taken as a separate sample. At inference time, each individual cough is converted to its mel-frequency spectrogram, then all spectrogram frames are stacked for individual coughs in the same episode to create one sample; this may be a combined cough. An individual cough may be included in the same episode if it occurs within 500 ms of the end of the previous individual cough.

Because the cough dataset was collected in-the-wild, the samples have significant background noise. To address this issue, data augmentation may be used. 4 extra copies of each cough with varying amplitude are first produced. The MUSAN dataset is used to apply background noise, music, and babble at 5 db to produce 15 total copies,

Speaker Dataset

To further reduce the impact of background noise, the speaker verification task is trained on the Voxceleb dataset. Voxceleb is a large-scale speaker verification dataset compiled from YouTube videos. Because many of the videos contain background noise, training the model on this dataset helps produce noise-robust features.

Training

Of the 16 users in the cough dataset, 12 are used for training and 4 are left out for test. Because the dataset is already small, the 3 users with more than 200 coughs are used for training, not test. User 4's samples are trained as they contain an abnormally high level of noise. Of the remaining 12 users to be used for test, 3-fold cross-validation may be used, holding out 4 users (2 male, 2 female) at a time.

For training, batches of size N users×M utterances are used, where N=12 and M=10, for both coughers and speakers. In comparison with the existing hyperparameters, examples described herein may also add an L2 regularization loss of 0.001 to prevent overfitting. 10,000 training steps are trained and the learning rate is decayed after 2,000 and 3,500 steps.

Results

Human Evaluation

To quantify the challenge of in-the-wild, natural tougher verification, a human baseline may be obtained. First, a human evaluator is permitted 5 minutes to listen to 5 random samples from each of the 12 test users so they can get familiar with listening to coughs. Then 4 users are selected from one of the cross-validation sets and perform verification and classification tests. For the verification test, each of the 4 users is iterated, 10 random combined coughs are selected as enrollment. The evaluator is able to listen to these 10 samples as often as they like while evaluating the test samples. 10 test samples may be randomly presented, 5 from the same user, 5 from different random users, and the evaluator may be asked to determine whether the cough is from the same user. For the classification test, 20 random samples of coughs may be presented, 5 from each of the 4 users, and the evaluator is asked to determine which of the 4 users each sample came from. In an example, 8 human evaluators are used and each performs both tests for the 3 folds. The results are listed in Table 2.

TABLE 2

| | FAR | FRR | Verif ACC | Class ACC |
|---|---|---|---|---|
| Metric | (%) | (%) | (%) | (%) |
| Average | 15.73 | 21.77 | 81.25 | 74.77 |
| Median | 12.07 | 16.38 | 83.89 | 77.69 |
| Std Dev | 7.34 | 3.95 | 7.59 | 13.72 |
| Best | 8.62 | 10.34 | 88.89 | 90.00 |
| Worst | 27.59 | 43.10 | 68.61 | 43.70 |

Statistics for the human evaluation including false accept rate (FAR), false reject rate (FRR), verification accuracy, and classification accuracy.

The results show evaluators performed better on the verification task. Evaluators commented that it was easier to make a binary decision (same speaker or different speaker) than a four-way classification. It may have also been easier for evaluators to use enrollment samples during the verification task when there were only 10 samples versus 40 for classification.

As demonstrated by the verification (81.25%) and classification (74.77%) accuracies, in-the-wild, natural tougher verification is a difficult task. Natural coughs are influenced by both environmental conditions and physiological changes that can produce dissimilar-sounding coughs. For example, a user may intentionally reduce the cough amplitude to not disturb a quiet setting. Or if they have a particularly challenging contaminant in their respiratory system, they may cough more harshly than usual. In-the-wild data collection also increases the difficulty as channel effects and background noise are more pronounced than in a controlled setting.

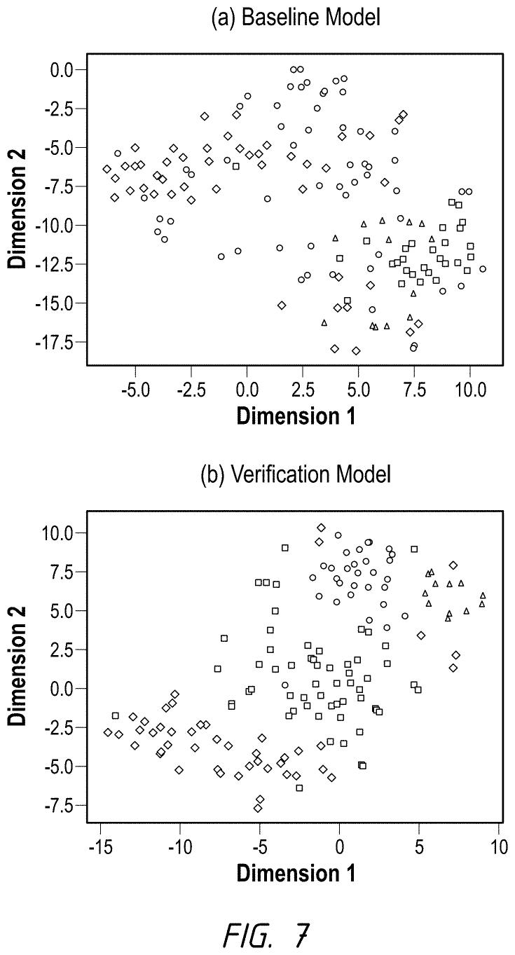

This is most easily viewed in FIG. 7 where (b) shows the t-SNE clustering of four users' cough embeddings using the model described herein. While most coughs by the same user are clustered together, there are outliers. The outliers sound very different from the rest of the cougher's samples. A human or model may not be able to classify these correctly.

Model Results

An example baseline model may have the same architecture and hyperparameters as the model described earlier, but is trained only for speaker verification on the Voxceleb dataset. To evaluate both the baseline model and the model described herein, 10 random samples per user may be used as enrollment. See Table 3 for the results.

TABLE 3

| | FAR | FRR | EER | Class ACC |
|---|---|---|---|---|
| Model | (%) | (%) | (%) | (%) |
| Human Evaluation | 15.73 | 21.77 | N/A | 74.77 |
| Baseline Model | 16.49 | 38.13 | 30.04 | 73.05 |
| Verification Model | 16.25 | 23.41 | 22.69 | 82.15 |

Results for the human evaluation, baseline model, and the verification model. EER refers to the equal error rate. For the baseline and the verification model, we find the verification model's similarity threshold that approximately matches the human evaluation FAR, then report the FRR at that threshold.

The results show the model described herein provides a 24.47% decrease in EER and a 12.46% increase in classification accuracy over the baseline. It also outperforms the human evaluators in the classification task on average by 9.87%.

Embedding Visualization

FIG. 7 shows the t-SNE clustering of the embeddings from one cross-validation fold. As shown in FIG. 7 the model described herein produces improved clusters of embeddings over the baseline model.

In the examples described herein, a novel multitask learning approach for in-the-wild, natural tougher verification is presented. Training with a secondary task of speaker verification helps overcome the small dataset problem to create a more general model. The model described herein can, on average, outperform human evaluators at a 4-way classification task using 10 enrollment samples. Using 3-fold cross-validation, a 22.69% EER and 82.15% classification accuracy is achieved.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

What is claimed is:

1. A method comprising:
recording an audio sample representative of a cough episode;
converting the audio sample to a spectrogram;
segmenting the spectrogram into frames;
processing the frames using a neural network to create a plurality of embeddings, wherein each of the plurality of embeddings corresponds with a respective one of the frames;
combining the plurality of embeddings to obtain a global embedding; and
predicting whether the audio sample is from an enrolled user based, at least in part, on the global embedding.

2. The method of claim 1, wherein combining the plurality of embeddings to obtain a global embedding comprises:
applying channel-wise average pooling to the plurality of embeddings to create an intermediate embedding for each of the short frames; and
combining the intermediate embeddings to obtain the global embedding.

3. The method of claim 2, wherein creating an intermediate embedding for each of the short frames utilizes a fully-connected layer of the neural network.

4. The method of claim 1, wherein the neural network is trained using a multi-task learning technique.

5. The claim of claim 4, wherein the multi-task learning technique comprises training on cough episodes and speech segments.

6. The method of claim 1, wherein the combining comprises averaging the plurality of embeddings.

7. The method of claim 1, wherein the predicting uses a cosine similarity metric.

8. The method of claim 1, wherein the neural network comprises a plurality of network nodes arranged in convolutional layers.

9. The method of claim 8, wherein each convolutional layer of the convolutional layers is followed by batch normalization (batch-norm) and a rectified linear unit (ReLu).

10. The method of claim 9, further comprising a skip connection between an output of a first convolutional layer's batch-norm and an output of a final convolutional layer's batch-norm.

11. The method of claim 1, further comprising:
enrolling a plurality of utterances from the enrolled riser by aggregating known utterances from the enrolled user; and
comparing the global embedding to the known utterances, wherein the predicting whether the audio sample is from the enrolled user is based, at least in part, on the comparison.

12. A non-transitory computer readable medium comprising instructions executable to cause a processor to:
receive coughs and test utterances from a plurality of individuals; and
train a neural network to generate a global embedding based on respective ones of the coughs and test utterances, wherein the global embedding is indicative of the individual corresponding to the respective coughs and test utterances, including:
perform speaker verification based on the test utterances; and
perform cougher verification based on the coughs.

13. The non-transitory computer readable medium of claim 12, wherein the instructions further cause the processor to:
convert the cough into a spectrogram; and
segment the spectrogram into a plurality of frames; and
process the plurality of frames to create an embedding; and
predict whether the individual is enrolled.

14. The non-transitory computer readable medium of claim 12, wherein the speaker verification is tested on a natural cough dataset comprising cough embeddings for a plurality of users.

15. The non transitory computer readable medium of claim 12, wherein the speaker verification is prioritized before the cougher verification.

16. A device comprising:
a microphone configured to receive an audio sample representative of a health sound audio;
at least one processor configured to create a global embedding based, at least in part, on the audio sample and predict a comparison result indicative of whether audio sample is from an enrolled user;
a display coupled to the processor and configured to display the comparison result.

17. The device of claim 16, wherein the processor is further configured to:
encode the health sound audio to obtain an alternate representation;
compare the alternate representation to enrolled health sound audio samples from known users; and
associate a selected one of the known users to the health sound audio based on the comparison.

18. The device of claim 17, wherein the processor is further configured to compare the global embedding to the enrolled health sound audio samples from the known users and predict whether the audio sample is from the enrolled user based, at least in part, on the comparison between the global embedding and the enrolled health sound audio samples.

19. The device of claim 16, further comprising:
a network communication interface configured to transmit the comparison result to a party.

20. The device of claim 16, wherein the processor comprises a neural network configured to utilize a network architecture to process the audio sample representative of a cough episode to create the global embedding,
wherein the network architecture comprises a plurality of layers including a convolutional layer and a final layer, and
wherein a skip connection links an output of the first convolution layer to an output of the final layer.
